(19) United States
(12) Patent Application Publication
(10) Pub. No.: US 2017/0266410 A1
(43) Pub. Date: Sep. 21, 2017

(54) MULTI-CHANNEL CATHETER INSERT

(71) Applicant: White Bear Medical LLC, White Bear Lake, MN (US)

(72) Inventors: Richard Farrell, Delwood, MN (US); Ryan Douglas, Stillwater, MN (US); Dennis Berke, River Falls, WI (US); Davis Flanagan, Saint Paul, MN (US); David Bontrager, Minneapolis, MN (US); Avong Lo, Brooklyn Park, MN (US); Brian Loushine, Minnetonka, MN (US)

(21) Appl. No.: 15/462,436

(22) Filed: Mar. 17, 2017

Related U.S. Application Data
(60) Provisional application No. 62/309,733, filed on Mar. 17, 2016.

(51) Int. Cl. A61M 25/00; A61B 5/00; A61M 1/00; A61B 1/07; A61M 25/01; A61M 25/06

(52) U.S. Cl. CPC A61M 25/0026; A61M 25/0147; A61M 25/0662; A61M 25/0009; A61M 25/0127; A61M 25/005; A61M 1/008; A61B 1/07; A61B 5/6852; A61M 2025/0004

(57) ABSTRACT

Apparatus and associated methods relate to a flexible extrusion having a number of radially extending members configured for slidable insertion into a lumen of a surgical catheter shaft. In an illustrative example, the extrusion may have a flexible wall and define an interior insert lumen extending along the longitudinal axis from a proximal end to a distal end. Each of the radially extending members may have a distal engaging surface. When the extrusion is slidably inserted, for example, into the lumen of the catheter shaft, the distal engaging surface of each of the plurality of radial extending members may slidably engage an interior surface of the catheter shaft. In some examples, the inserted extrusion may define an annular distribution of longitudinally extending channels between a proximal and a distal end of the catheter. The slidable construction may advantageously simplify assembly, for example. The channels may offer end-to-end communication.

### CROSS-REFERENCE TO RELATED APPLICATIONS

This application claims the benefit of U.S. Provisional Application Ser. No. 62/309,733, titled “Catheter Shaft for High Transfer of Torque,” filed by Farrell, et al., on Mar. 17, 2016.

This application incorporates the entire contents of the foregoing application(s) herein by reference.

### TECHNICAL FIELD

Various embodiments relate generally to construction of minimally invasive catheters, including, for example, steerable catheters.

### SUMMARY

Apparatus and associated methods relate to a flexible extrusion having a number of radially extending members configured for slidable insertion into a lumen of a surgical catheter shaft. In an illustrative example, the extrusion may have a flexible wall and define an interior insert lumen extending along the longitudinal axis from a proximal end to a distal end. Each of the radially extending members may have a distal engaging surface. When the extrusion is slidably inserted, for example, into the lumen of the catheter shaft, the distal engaging surface of each of the plurality of radial extending members may slidably engage an interior surface of the catheter shaft. In some examples, the inserted extrusion may define an annular distribution of longitudinally extending channels between a proximal and a distal end of the catheter. The slidable construction may advantageously simplify assembly, for example. The channels may offer end-to-end communication.

Various embodiments may achieve one or more advantages. For example, some embodiments may be advantageously assembled at a reduced labor, time, and expense by sliding the insert into the outer sheath. One or more conventional assembly steps may be reduced or eliminated in a catheter construction process. In operation, the catheter can employ the channels for an array of functional filaments or to communicate flowable media between the opposing ends of the catheter, for example. Various embodiments may further be capable of being partially assembled with the MCCI insert and preloaded with one or more steering wire sets, for example. In some embodiments, partially assembled MCCI assemblies may be stored in inventory, and subsequently customized by outfitting with selected filaments or, in operation, communicating selected media, as needed for custom surgical applications, for example.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts a schematic diagram representing a surgical application of a catheter constructed with an exemplary Multi-Channel Catheter Insert (MCCI).

FIGS. 2A, 2B and 2C depict perspective and end views of an exemplary MCCI.

FIGS. 3A, 3B, and 3C depict cross-sectional and perspective views of exemplary configurations of an MCCI.

FIGS. 4A, 4B, 4C depict perspective views of exemplary configurations of an MCCI prepared for insertion and pre-loaded with two or four sets of steering wires.

Like reference symbols in the various drawings indicate like elements.

### DETAILED DESCRIPTION OF ILLUSTRATIVE EMBODIMENTS

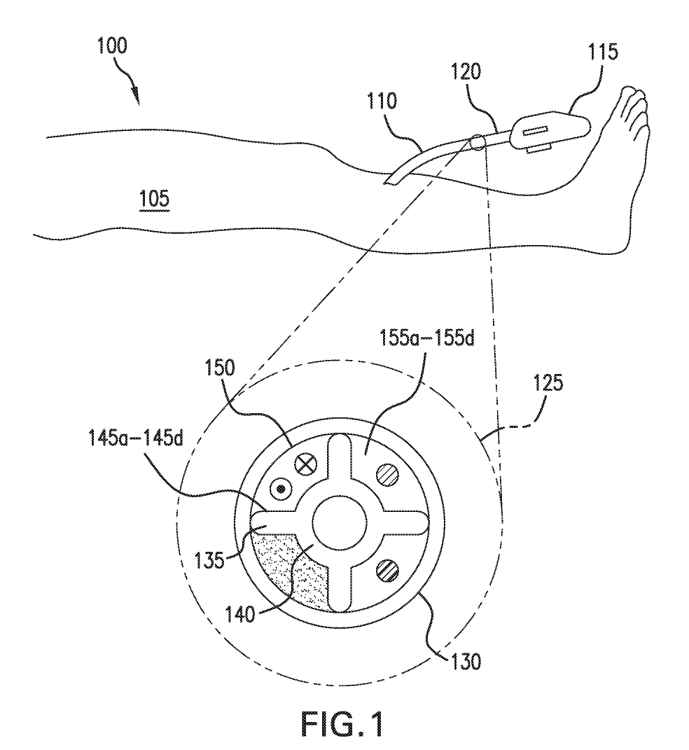

FIG. 1 depicts a schematic diagram representing a surgical application of a catheter constructed with an exemplary Multi-Channel Catheter Insert (MCCI). In the depicted figure, a surgical operation 100 on a patient 105 is performed using a minimally invasive catheter 110. The catheter 110 includes a handle 115, which may be controlled by a surgeon, and a shaft 120 that is extending from the handle 115 while being deployed subcutaneously into the patient 105. In a magnified cross-section 125, the shaft 120 includes an outer sheath 130 and an insert 135. The insert 135 includes an insert wall 140 and a number of members 145a-145d that radially extend from the wall 140. Each of the members 145a-145d slidably engage an inner surface 150 of the outer sheath 130. In the space between the inner surface 150 and the insert wall 140, the members 145a-145d define an annular distribution of longitudinally extending channels 155a-155d extending between opposing ends of the shaft 120. The catheter 110 can be advantageously assembled at a reduced labor, time, and expense by sliding the insert 135 into the outer sheath 130. In operation, the catheter 110 can employ the channels 155a-155d for an array of functional filaments or to communicate flowable media between the opposing ends of the catheter 110.

The channels 155a-155d, when slidably engaged with an inner surface 150, define tangentially distributed, isolated channels that run longitudinally along the length of the shaft 120.

Within one or more of these channels 155a-155d, one or more functional filaments may be employed. The functional filaments may include, for example, one or more distal tip steering wires, electrical wires, or fiber optics. The steering wires may be constructed of, for example, polymer or a non-reactive metal. In the depicted FIG. 1, one of the channels 155a-155d includes a pair of wires, one conducting a current in a distal direction (e.g., showing a dot indicating current flow out of the page) and one conducting the current in a proximal direction (e.g., showing an X indicating current flow into the page). The pair of wires may be conducting power and/or signals to or from a sensor proximate the distal tip.

Moving clockwise, an adjacent one of the channels 155a-155d includes a steering pull wire, which may be secured, for example, to a distal end of the shaft 120. The pull wire may be tensioned from a proximal end, for example with the handle 115, to deflect the tip such that, in combination with any needed rotation, may be used to position the distal tip in a required orientation (e.g., for steering, to deliver a therapy, for diagnostic sensing).

Moving clockwise again, an adjacent one of the channels 155a-155d includes an optical fiber. In some examples, one or more optical fibers may be used to direct light signals to or from the distal tip (e.g., to deliver therapy, for illumination, for optical visualization).

Moving clockwise once again, an adjacent one of the channels 155a-155d includes a flowable media, e.g. a cooling gas, that can be communicated between the proximal and distal ends of the shaft 120. In some implementations, one or more of the channels 155a-155d may provide a substantially sealed passageway suitable to convey flowable media (e.g., low pressure gasses) between the opposing ends of the shaft 120.

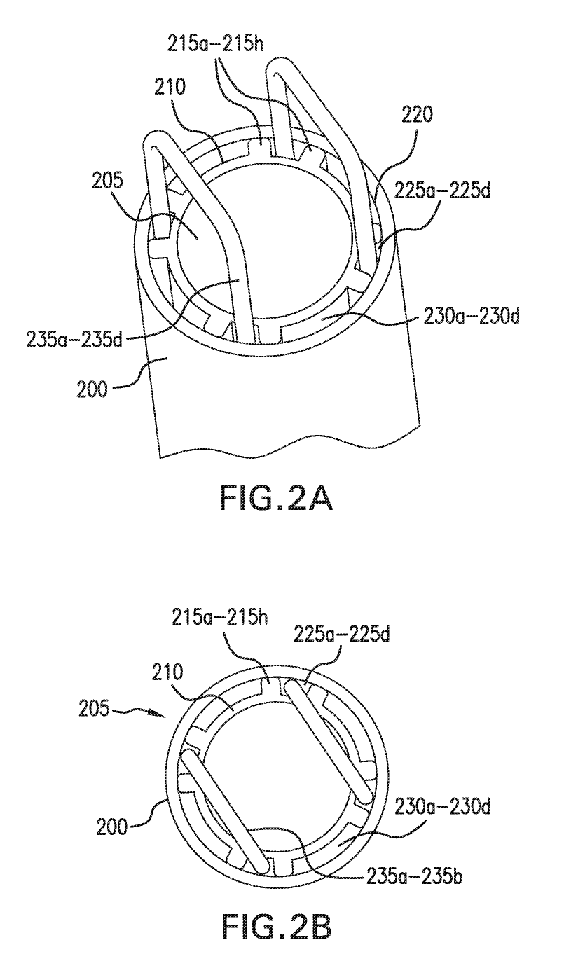

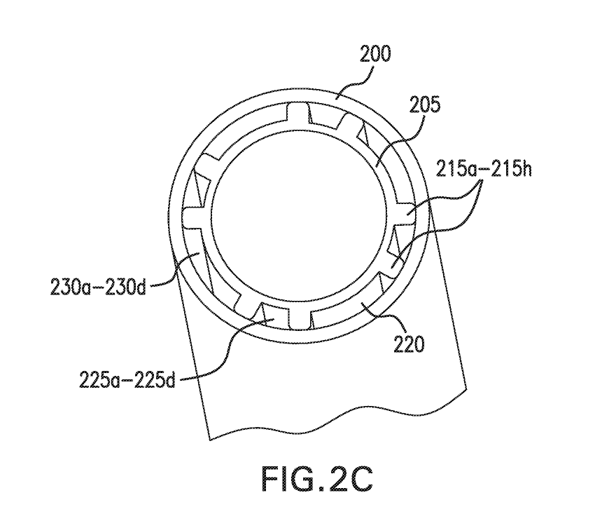

FIGS. 2A, 2B and 2C depict perspective and end views of an exemplary MCCI. In the depicted Figures, a perspective view of a catheter includes an outer sheath 200 and an insert 205. The insert 205 includes an insert wall 210 and a number of members 215a-215h radially extending from the insert wall 210 to the inner surface 220 of the outer sheath 200. Each of the members 215a-215h slidably engages the inner surface 220 of the outer sheath 200. The members 215a-215h are annularly distributed to create a number of narrower longitudinal channels 225a-225d and a number of wider longitudinal channels 230a-230d. The catheter 110 employs the narrower longitudinal channels 225a-225d for two sets of slidable steering wires 235a-235b.

In the depicted FIG. 2B, an end view of the insert 205 includes an end view of the outer sheath 200 and of the insert 205 with the two sets of steering wires 235a-235b threaded through the narrower longitudinal channels 225a-225d. In various embodiments, the threaded installation of the steering wires 235a-235b through the longitudinal channels 225a-225d may advantageously reduce labor, time, and expense in construction of a surgical catheter, such as the catheter 110 of FIG. 1.

In the depicted FIG. 2C, a perspective view of a catheter shaft includes the outer sheath 200 assembled with the insert 205. The insert 205 includes the insert wall 210 and eight members 215a-215h radially extending from the insert wall 210 to the inner surface 220 of the outer sheath 200. Each of the members 215a-215h slidably engages the inner surface 220 of the outer sheath 200. The members 215a-215h are annularly distributed to create the narrower longitudinal channels 225a-225d and the wider longitudinal channels 230a-230d. The catheter 110 is advantageously constructed with narrower longitudinal channels 225a-225d and the wider longitudinal channels 230a-230d to provide a number of physically separate lumens for various filaments (e.g., wires, steering wires, optical fibers, electrical) and/or to optimize for conveying flowable media between opposing ends of the catheter.

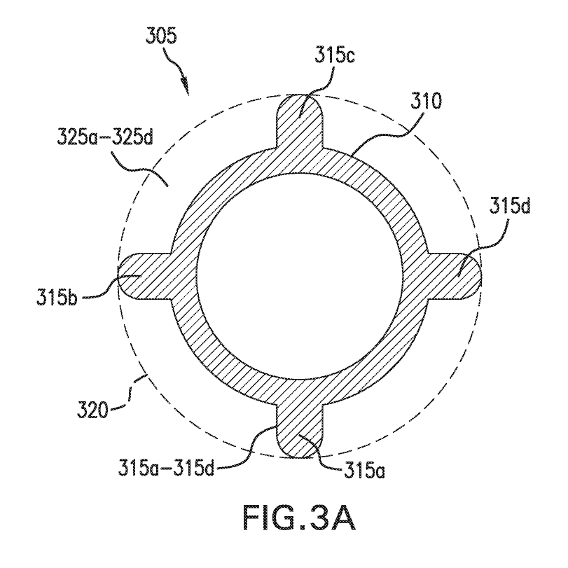

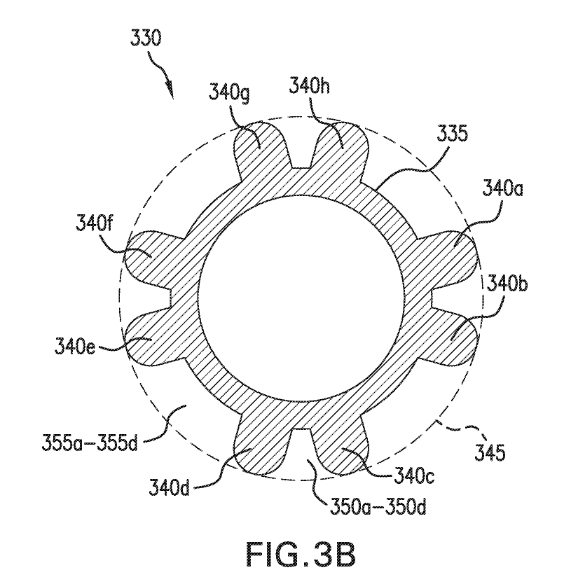

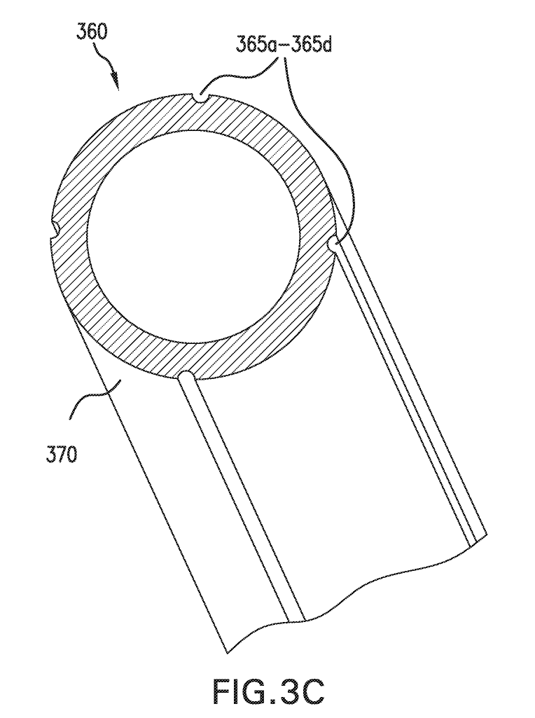

FIGS. 3A, 3B, and 3C depict cross-sectional and perspective views of exemplary configurations of an MCCI. In FIG. 3A, a cross-sectional view depicts a catheter insert 305. The insert 305 includes an insert wall 310 and four radially extending members 315a-315d extending from the insert wall 310 to the inner surface of the outer sheath 320. The radially extending members 315a-315d slidably engage the inner surface of the outer sheath 320. The radially extending members 315a-315d are annularly distributed to create a number of longitudinal channels 325a-325d, whereby the longitudinal channels 325a-325d extend between opposing ends.

In FIG. 3B, a cross-sectional view depicts a catheter insert 330. The insert 330 includes an insert wall 335 and between eight radially extending members 340a-340h extending from the insert wall 335 to the inner surface of the outer sheath 345. The radially extending members 340a-340h slidably engage the inner surface of the outer sheath 345. The radially extending members 340a-340h are annularly distributed to create a number of narrower longitudinal channels 350a-350d and a number of wider longitudinal channels 355a-355d. The narrower longitudinal channels 350a-350d and the wider longitudinal channels 355a-355d extend longitudinally between opposing ends.

In FIG. 3C, a perspective view depicts a catheter insert 360. The insert 360 includes four longitudinally extending grooves 365a-365d formed in a cylindrically shaped body of the insert 360. When the insert 360 is inserted into an outer sheath, the grooves 365a-365d may define four isolated longitudinal channels between an inner surface of an outer sheath and the body of the insert 360. The outer surface 370 of the insert 360 slidably engages with an inner surface of an outer sheath.

The exemplary configurations in the cross-sectional and perspective views of FIGS. 3A, 3B, and 3C may advantageously be constructed to employ various filaments and to convey flowable media between opposing ends through any of the various longitudinal channels.

The exemplary configurations in FIGS. 3A, 3B, and 3C depict various longitudinal channels or grooves each optimally constructed for various catheter functions. The functions, for example, may include collecting information, aspirating, or conveying flowable media. Some embodiments may be constructed employing a combination of the longitudinally extending grooves 365a-365d and the longitudinally extending channels 325a-325d constructed of the radially extending members 315a-315d. Some embodiments may, for example, perform various differing functions at one time as the longitudinal channels or grooves depicted in the FIGS. 3A, 3B, and 3C may be isolated from one another and from may be isolated from a central lumen. The exemplary configurations in the cross-sectional and perspective views of FIGS. 3A, 3B, and 3C may advantageously reduce costs, labor, and time for constructing a catheter capable of multiple functionality.

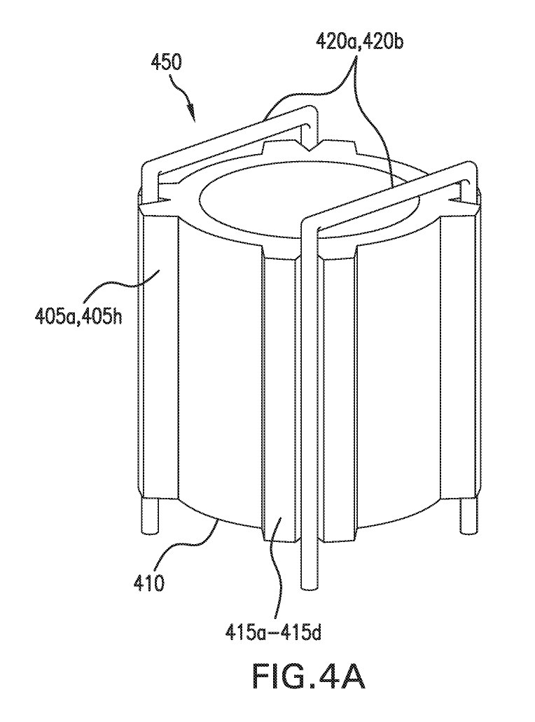

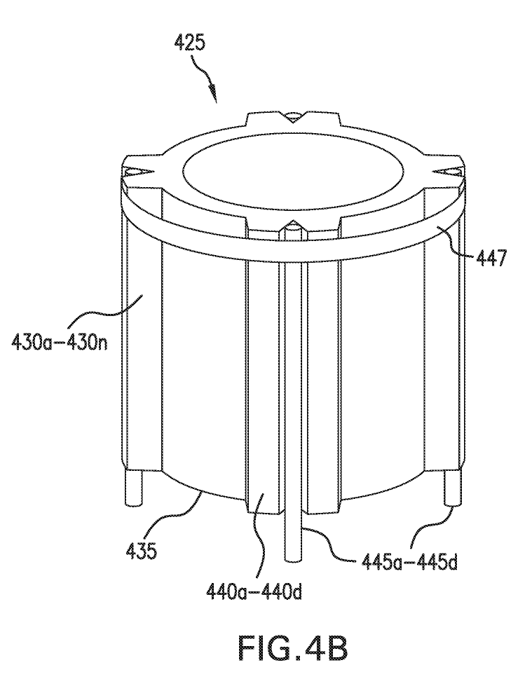

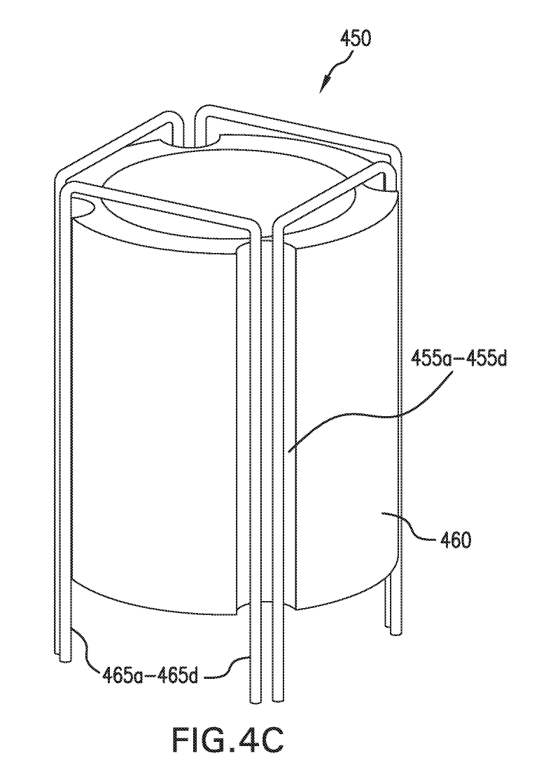

FIGS. 4A, 4B, and 4C depict perspective views of exemplary configurations of an MCCI prepared for insertion and pre-loaded with two or four sets of steering wires.

FIG. 4A depicts a perspective view of an insert 400. The insert 400 includes a number of radially extending members 405a-405h extending from longitudinally spaced annular rings that form a wall 410. In the spaces between the rings, the wall is open. The radially extending members 405a-405h are annularly distributed to create a number of narrower longitudinal channels 415a-415d loaded with threaded steering wires 420a-420b.

FIG. 4B depicts a perspective view of an insert 425. The insert 425 includes a number of radially extending members 430a-430h extending from an insert wall 435. The radially extending members 430a-430h are annularly distributed to create a number of longitudinal channels 440a-440d. The longitudinal channels 440a-440d contain steering wires 445a-445d extending from a proximal end of the insert 425 and attached or fastened near a distal end of the insert 425. In some embodiments, the wires 445a-445d may be soldered or otherwise fastened to a metallic ring 447 disposed near a distal end of the catheter shaft.

FIG. 4C depicts a perspective view of an insert 450. The insert 450 includes a number of longitudinally extending channels 455a-455d along an insert wall 460. The longitudinally extending channels 455a-455d contain four sets of threaded steering wires 465a-465d, which may provide four directions of steering (e.g., left, right, up, and down).

In some embodiments, the various wires may be threaded and the various wires may not be fixed or fused to any portion of an insert or of an outer sheath, thereby advantageously decreasing the cost, labor, and time for constructing and employing these embodiments. In some embodiments, the various wires may be fixed or fused to various points of an insert or at various points along an outer sheath, advantageously increasing functionality. The various wires may be fixed or fused to various points employing, for example, lasers, heat, or magnets. In the depicted embodiments in FIGS. 4A, 4B, and 4C, pre-loading and pre-installing the wires, whether threaded or fixed to various points, may advantageously decrease the costs, labor, and time for constructing a catheter and employing a catheter for various functionality.

In some embodiments, as depicted in FIG. 4A, the insert may be constructed as various insert rings to which the radially extending members are attached and are annularly distributed around. The radially extending members may form columns constructed to extend from the insert rings extending longitudinally and connecting the various insert rings to create various isolated, longitudinally extending channels. This embodiment creates an advantageously more open structure that may be used for various functionality constructed at less cost, time or requiring less labor.

In some embodiments, an insert wall or an insert ring may be constructed of or supported by various helically wound coils. An insert wall or an insert ring supported by various helically wound coils may be a separate construction with which an inner insert wall or an inner insert ring wall slidably engages. An insert wall or an insert ring supported by various helically wound coils may be pre-installed for advantageous construction of a catheter employing multiple functions. In some embodiments, an insert wall may be constructed of various helically wound coils. An insert wall or an insert ring constructed of or supported by various helically wound coils may have advantageous steerability and rigidity features.

Although various embodiments have been described with reference to the Figures, other embodiments are possible. For example, one or more of the channels may have available space for providing additional functionality in addition to steering of the distal end. Various implementations my use available channels for delivering and/or withdrawing (e.g., aspiration) materials, energy, and/or signals between the distal and proximal ends of the catheter.

Referring to FIG. 1, various assembly techniques may be used to install the functional filaments into the channels 155a-155d to form the catheter 110.

In some embodiments, the functional filaments may be threaded through the channels 155a-155d after insertion of insert 135 into the outer sheath 130 to form the catheter 110.

In some embodiments, inserting a filament in one of the available channels after the MCCI has been inserted into the outer sheath may be accomplished using a magnetic field source and a magnetically permeable leader that releasably attaches to an end of the filament. Upon using an external magnetic field to urge the magnetically permeable leader through one of the channels 155a-155d, from a proximal end to a distal end (or vice versa) of the catheter shaft 120, the leader may be disconnected from the filament, leaving the filament in place along the entire length of one of the channels 155a-155d. In some implementations, the leader may be, for example a re-usable clam shell-style. In some embodiments, the leader may be releasably engaged to the filament by a frictional coupling. In some embodiments, the leader may be urged against a knot formed in the lead end of the filament. In an automated assembly step, a conveyor system may provide relative motion, along the length of the catheter shaft 120, between the MCCI and a magnetic field source (e.g., permanent magnet, electro magnet) that is configured to urge a magnetically permeable leader. When a leader is positioned at one end of one of the channels 155a-155d, the conveyor may impart relative motion between the magnet source and the outer sheath 130 while attracting the leader to remain in close proximity to the magnet source, thereby urging the leader through the longitudinal length of one of the channels 155a-155d. This may automate the process of threading a selected filament into one of the channels 155a-155d after insertion of the insert 135 into the outer sheath 130.

In some embodiments, the functional filaments may be threaded through the channels 155a-155d during insertion of insert 135 into the outer sheath 130 to form the catheter 110.

For example, the filament may be installed in the catheter 110 by attaching the filament to the insert 135 prior to the insert 135 being inserted into the outer sheath 130. In some implementations, the filament may be fixed to a point on the outer sheath 130 near one end, for example, using a weld point. Such a weld point may be made to a metal or plastic ring, for example. The insert 135 may be inserted into the outer sheath 130 at the end proximate the weld point.

In some implementations, during insertion of the insert 135, a filament may, for example, wrap across a lead insertion end of the insert 135. As the insertion is performed, additional length of filament is drawn into two of the channels, as required during the insertion, from a filament supply source and/or from a predetermined length of the filament. In an example, the resulting loop of inserted filament residing in two of the channels 135a-135d may provide a set of steering pull wires for steering the distal tip by applying tension on one or both of the wires using, for example, the catheter handle 115.

In some embodiments, the insert 135 may be pre-installed in the outer sheath 130 prior to insertion of the insert 135.

The various elements used to construct the catheter 110 may allow for advantageously constructing a catheter for various purposes at a reduced labor, time, and expense by construction using a sliding insert, such as the insert 135. Various embodiments may further provide for flexible, on-demand customization of functionality by installing functional filaments according to a customized application to upgrade a supply of one or more catheters pre-assembled with an MCCI.

In an example, the filament to be installed into a selected one of the channels may be a fiber optic thread, which may be used to deliver light to illuminate a distal surface proximate the end of the catheter. In some embodiments, the filament employed in a selected channel may be an imaging technology, which may be used to map a catheter pathway or capture a still or moving image from a distal end of a catheter.

In some implementations, a filament may conduct one or more electrical signals. The various electrical signals may be conveyed via the multiple channel construction of a catheter. One or more channels of various widths may be employed to convey various electrical filaments. These implementations may be combined with other embodiments due to the multiple channel construction of the insert.

In some embodiments, at least one isolated available channel after the MCCI has been inserted into an outer sheath may be employed to convey flowable media. A flowable media may include, for example, gas, liquid, or an alternative fluid. These flowable media may either be delivered into a patient or may be withdrawn (e.g. aspirated) from a patient. An isolated channel may employ a filtering medium (e.g., charcoal or insulation batting) through which to convey a flowable media. In some embodiments, an isolated channel employed to convey flowable media may also employ at least one filament to convey information. The filament employed within the same channel as the flowable media may, for example, convey information relating to the flowable media or the procedure.

In some implementations, at least one isolated channel or an inner lumen may be employed to deliver or withdraw solid media or structure. The delivery of solid media may be, for example, a valve or a stent.

In various embodiments, the number of radially extending members may be between 2 and 20, such as for example at least 4 and not more than 6, at least 8 and not more than 14, or at least 14 and not more than 20.

In some embodiments, a catheter may be constructed by installing a high torque transmission structure coaxially with an MCCI. An example of a high torque transmission structure that may be employed in conjunction with an MCCI is described, for example, with reference to at least FIGS. 2 and 6 of U.S. Provisional Application Ser. No. 62/309,733, titled “Catheter Shaft for High Transfer of Torque,” filed by Farrell, et al., on Mar. 17, 2016, which is incorporated by reference in its entirety herein.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components. Accordingly, other implementations are within the scope of the following claims.

What is claimed is:

1. A steerable catheter apparatus comprising:
an outer sheath having a flexible cylindrical wall and defining an interior sheath lumen extending along a longitudinal axis from a proximal end to a distal end; and,
an insert module adapted to slidably insert into the interior lumen of the outer sheath, the insert module having a flexible wall and defining an interior insert lumen extending along the longitudinal axis from the proximal end to the distal end, wherein the insert module further comprises a plurality of radially extending members, each of the radially extending members having a distal engaging surface,
wherein, when the insert module is slidably inserted into the interior sheath lumen, the distal engaging surface of each of the plurality of radial extending members slidably engages an interior surface of the outer sheath cylindrical wall, thereby defining an annular distribution of a plurality of longitudinally extending channels between the proximal end and the distal end.

2. The steerable catheter apparatus of claim 1, wherein the insert module is formed by an extrusion process.

3. The steerable catheter apparatus of claim 1, wherein the insert module wall is substantially stiff along the longitudinal axis.

4. The steerable catheter apparatus of claim 3, wherein the outer sheath wall and the insert module wall are substantially flexible along a lateral axis.

5. The steerable catheter apparatus of claim 1, wherein the outer sheath wall is substantially stiff along the longitudinal axis.

6. The steerable catheter apparatus of claim 5, wherein the outer sheath wall and the insert module wall are substantially flexible along a lateral axis.

7. The steerable catheter apparatus of claim 6, wherein at least one longitudinally extending channel conveys at least one steering mechanism.

8. The steerable catheter apparatus of claim 7, wherein the steering mechanism is a steering wire.

9. The steerable catheter apparatus of claim 1, further comprising a first steering wire that extends continuously from the proximal end to the distal end in a first one of the longitudinally extending channels, from the distal end of the first longitudinally extending channel to a distal end of a second one of the longitudinally extending channels, and from the distal end to the proximal end in the second one of the longitudinally extending channels.

10. The steerable catheter apparatus of claim 9, further comprising a second steering wire that extends continuously from the proximal end to the distal end in a third one of the longitudinally extending channels, from the distal end of the third longitudinally extending channel to a distal end of a fourth one of the longitudinally extending channels, and from the distal end to the proximal end in the fourth one of the longitudinally extending channels.

11. A multiple channel catheter insert (MCCI) apparatus comprising:
an insert module adapted to slidably insert into an interior lumen of an outer sheath having a flexible cylindrical wall and defining an interior sheath lumen extending along a longitudinal axis from a proximal end to a distal end, the insert module having a flexible wall and defining an interior insert lumen extending along the longitudinal axis from the proximal end to the distal end, wherein the insert module further comprises a plurality of radially extending members, each of the radially extending members having a distal engaging surface,
wherein, the insert module is configured to be slidably inserted into the interior sheath lumen such that the distal engaging surface of each of the plurality of radial extending members is adapted to slidably engage an interior surface of the outer sheath cylindrical wall to define an annular distribution of a plurality of longitudinally extending channels between the proximal end and the distal end.

12. The multiple channel catheter insert (MCCI) of claim 11, wherein the insert module is formed by an extrusion process.

13. The multiple channel catheter insert (MCCI) of claim 11, wherein the insert module wall is substantially stiff along the longitudinal axis.

14. The multiple channel catheter insert (MCCI) of claim 11, wherein the plurality of radially extending members is at least 8 and not more than 14.

15. The multiple channel catheter insert (MCCI) of claim 11, wherein the plurality of radially extending members is at least 4 and not more than 6.

16. The multiple channel catheter insert (MCCI) of claim 11, further comprising a first steering wire that extends continuously from the proximal end to the distal end in a first one of the longitudinally extending channels, from the distal end of the first longitudinally extending channel to a distal end of a second one of the longitudinally extending channels, and from the distal end to the proximal end in the second one of the longitudinally extending channels.

17. The multiple channel catheter insert (MCCI) of claim 16, further comprising a second steering wire that extends continuously from the proximal end to the distal end in a third one of the longitudinally extending channels, from the distal end of the third longitudinally extending channel to a distal end of a fourth one of the longitudinally extending channels, and from the distal end to the proximal end in the fourth one of the longitudinally extending channels.

18. A steerable catheter apparatus comprising:
an outer sheath having a flexible cylindrical wall and defining an interior sheath lumen extending along a longitudinal axis from a proximal end to a distal end; and,
means for defining an annular distribution of a plurality of longitudinally extending channels between the proximal end and the distal end.

19. The steerable catheter apparatus of claim 18, further comprising a first steering wire that extends continuously from the proximal end to the distal end in a first one of the longitudinally extending channels, from the distal end of the first longitudinally extending channel to a distal end of a second one of the longitudinally extending channels, and from the distal end to the proximal end in the second one of the longitudinally extending channels.

20. The steerable catheter apparatus of claim 19, further comprising a second steering wire that extends continuously from the proximal end to the distal end in a third one of the longitudinally extending channels, from the distal end of the third longitudinally extending channel to a distal end of a fourth one of the longitudinally extending channels, and from the distal end to the proximal end in the fourth one of the longitudinally extending channels.
